(19) United States
(12) Patent Application Publication
(10) Pub. No.: US 2021/0341496 A1
(43) Pub. Date: Nov. 4, 2021

(54) Systems and Methods for Detecting or Monitoring Insulin

(71) Applicant: The Florida State University Research Foundation, Inc., Tallahassee, FL (US)

(72) Inventors: Michael G. Roper, Tallahassee, FL (US); Joel E. Adablah, Tallahassee, FL (US); Matthew Donohue, Tallahassee, FL (US)

(21) Appl. No.: 17/246,546

(22) Filed: Apr. 30, 2021

Related U.S. Application Data
(60) Provisional application No. 63/018,845, filed on May 1, 2020.

(51) Int. Cl. G01N 33/74; C07K 16/26; G01N 33/84; B01L 3/00; G01N 21/64

(52) U.S. Cl. CPC G01N 33/74; C07K 16/26; B01L 2200/027; B01L 3/5027; G01N 21/6445; G01N 33/84

(57) ABSTRACT

Methods and systems for insulin detection. The methods may include contacting one or more islets with glucose to produce a first stream that is then contacted with an anti-insulin antibody and a labeled insulin to produce a second stream. The second stream may be analyzed to determine a ratio of antibody-bound (B) labeled insulin to free (F) labeled insulin in the second stream, wherein the ratio of B:F is inversely related to a concentration of target insulin.

### CROSS-REFERENCE TO RELATED APPLICATIONS

This application claims priority to U.S. Provisional Patent Application No. 63/018,845, filed May 1, 2020, which is incorporated herein by reference.

### STATEMENT REGARDING FEDERALLY SPONSORED RESEARCH OR DEVELOPMENT

This invention was made with government support under Contract Nos. 1UC4DK116283-01 and 2R01DK080714 awarded by the National Institutes of Health. The government has certain rights in this invention.

### BACKGROUND

Efforts have been made to understand the microstructure and innervation of the pancreas and its endocrine fraction, the islets of Langerhans. Insulin release from single islets is pulsatile, typically with a period of 3-7 minutes, and similar periods have been reported in vivo when insulin levels are measured in the portal vein. Disordered hormone oscillations have been observed in individuals prior to and during Type 2 diabetes and the liver may be more receptive to pulsatile rather than static insulin levels. For an oscillatory insulin profile, the release of insulin from a large majority of islets within the pancreas should be synchronized.

One potential mechanism of how islets may be synchronized is by continuous resetting of islet oscillations due to repeated pulses of acetylcholine (ACh) from parasympathetic ganglia, clusters of autonomic nerve cell bodies that are interspersed throughout and innervate the pancreas. ACh potentiates glucose-stimulated insulin secretion (GSIS) by activating M₃-muscarinic receptors (M3R) in islets. This activation leads to mobilization of Ca²⁺ from inositol-1,4,5-trisphosphate (IP₃)-sensitive Ca²⁺ stores in the endoplasmic reticulum (ER) and the acceleration of protein kinase C-mediated insulin exocytosis.

This explanation of islet synchronicity has been supported by the observation of spontaneous bursts of electrical activity in ganglia of a feline pancreas every 6-8 minutes, correlating with the insulin oscillation periods found in plasma. In addition, insulin oscillations at a similar period have been observed during ex vivo perfusion of the pancreas, implying the synchronizing agent must be local to the pancreas. In vitro studies have also shown that a single pulse of ACh or the M3R agonist, carbachol (CCh), can promote transient synchronization in groups of murine islets in a glucose-rich environment. Groups of murine islets exposed to periodic or aperiodic pulses of CCh can be synchronized. However, the only evidence reported has been the synchronization of intracellular Ca²⁺ oscillations as a marker of insulin release with no report demonstrating synchronized hormone release.

The most established methods for insulin measurement are heterogeneous antibody-based assays, and chief among these are enzyme-linked immunosorbent assays (ELISAs). These heterogeneous assays have high sensitivity, yet typically require multiple wash steps forcing the assays to be offline due to their long processing times. Other types of immunoassays have been developed to limit these drawbacks, for example, by incorporating electrophoretic immunoassays into microfluidic systems. While this type of analysis has many advantages over ELISA, including the ability to perform online measurements, there are several drawbacks to the method that limit its incorporation into other laboratories, such as working with microfluidic channel dimensions less than 10 μm in depth. These small channel dimensions typically are used to reduce the electric current and subsequent Joule heating to acceptable levels, but can also lead to more arduous channel fabrication processes and/or higher risks of clogging.

In contrast, homogeneous immunoassays do not require a wash or separation step, thereby easing the requirements for the channel dimensions. Several homogeneous based approaches for insulin quantitation have been described, with only a few being utilized for online measurement of insulin release in microfluidic systems (Schrell, A. M. et al. Anal. Methods 2017, 9 (1), 38-45; Glieberman, A. L. et al. Lab Chip 2019, 19 (18), 2993-3010). Fluorescence anisotropy (or fluorescence polarization) immunoassays have been used for detection of antibodies or antigens in biological samples.

Fluorescence anisotropy is an all-optical, quantitative method for analysis of the degree of rotational depolarization of a fluorophore. This technique (or a similar technique, fluorescence polarization) has been used for quantitative measurements of homogeneous immunoassays, typically in plate reader-based systems. A fluorescence anisotropy immunoassay for quantification of insulin release from islets using Cy5-insulin as the labeled Ins* and gravity as the means to drive fluid flow in the device has been previously reported (Schrell, A. M. et al. Anal. Methods 2017, 9 (1), 38-45).

Attempts to measure dynamic insulin release from pancreatic islets using fluorescence anisotropy have been made, but none has been able to show rapid insulin dynamics from single or multiple islets. Additionally, rapid flow switches for delivery of complex reagent patterns to islets were not feasible. Assay dynamic ranges and signal-to-noise were limited by their fluorescent dye. (see, e.g., Analytical Methods, 2017, 9, 38-45; Lab on a Chip, 2019, 19, 18, 2949-3142).

There remains a need for methods of detecting or monitoring a target, such as insulin, that avoid commonly used heterogeneous formats, such as enzyme-linked immunosorbent assays (ELISA), which typically are time consuming, are labor intensive, require complex antibody incubations and/or washes, or a combination thereof.

### BRIEF SUMMARY

Provided herein are fluorescence anisotropy-based homogeneous assays and methods that may overcome one or more of the foregoing disadvantages of heterogeneous formats. The methods provided herein include methods for detecting or monitoring, including online monitoring, of insulin from pancreatic islets. In some embodiments, the use of SeTau-647 as the fluorescent label for a labeled insulin (Ins*) provides a larger dynamic range to the insulin assay compared to Cy5. In some embodiments, an actively-controlled perfusion system improves the robustness of the devices herein and results in an increased temporal resolution of the assay.

In one aspect, devices and systems that may be used for detecting or monitoring a target, such as insulin, are provided. In some embodiments, the systems include a microfluidic device, wherein the microfluidic device includes an islet chamber, and (i) a first fluidic inlet upstream of the islet chamber, (ii) a second fluidic inlet upstream of the islet chamber, (iii) a third fluidic inlet downstream of the islet chamber, and (iv) a fourth fluidic inlet downstream of the islet chamber; a metal plate defining one or more voids, wherein the microfluidic device is arranged on the metal plate, and the one or more voids are configured to permit optical detection of a stream within a channel of the microfluidic device; a first fluid reservoir pressurized with a first flow controller, wherein the first fluid reservoir is in fluid communication with the fourth fluidic inlet.

In another aspect, methods for detecting or monitoring a target, such as insulin, are provided. In some embodiments, the methods include providing a sample that includes an islet; contacting the sample with (i) glucose or (ii) glucose and a balanced salt solution to produce a first stream that includes a target insulin; contacting the first stream with an anti-insulin antibody and a labeled insulin to produce a second stream that includes the target insulin, an amount of an antibody-bound (B) labeled insulin, and an amount of free (F) labeled insulin; and determining a ratio of the amount of the antibody-bound (B) labeled insulin to the amount of the free (F) labeled insulin in the second stream, wherein the ratio of B:F is inversely related to a concentration of the target insulin in the second stream.

In some embodiments, the methods include performing a fluorescence anisotropy-based homogeneous assay of a liquid that includes a labeled target, wherein the labeled target is labeled with a squaraine rotaxane fluorophore.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1A depicts an embodiment of a microfluidic device.

FIG. 1B depicts an embodiment of a system.

FIG. 2 depicts a schematic of an embodiment of an active pressure-drive perfusion system.

FIG. 3A depicts a schematic of an embodiment of a microfluidic chip.

FIG. 3B depicts the results of a flow characterization experiment.

FIG. 3C depicts a profile of programmed flow rates (right axis) of a solution delivered into the microfluidic device of FIG. 3A from inlet 2, and the fluorescence signal (left axis) measured by a photomultiplier tube as a function of time at the islet chamber during pulsing of the solution.

FIG. 4 depicts an embodiment of an optical setup that may be used to measure fluorescence anisotropy.

FIG. 5A depicts calculated immunoassay calibration curves for embodiments of fluorophores.

FIG. 5B depicts a comparison of experimentally-obtained calibration curves.

FIG. 6A depicts the results of a glucose-stimulated insulin secretion test of an embodiment of a single islet.

FIG. 6B depicts the results of a glucose-stimulated insulin secretion test of an embodiment of grouped islets.

FIG. 7A depicts the results of a glucose-stimulated insulin secretion test of an embodiment of a single islet.

FIG. 7B depicts the results of a glucose-stimulated insulin secretion test of an embodiment of a single islet.

FIG. 7C depicts the results of a glucose-stimulated insulin secretion test of an embodiment of a single islet.

FIG. 8A depicts the results of a glucose-stimulated insulin secretion test of an embodiment of grouped islets.

FIG. 8B depicts the results of a glucose-stimulated insulin secretion test of an embodiment of grouped islets.

FIG. 8C depicts the results of a glucose-stimulated insulin secretion test of an embodiment of grouped islets.

FIG. 9 depicts the results of a glucose-stimulated insulin secretion test of embodiments of three healthy human islets.

FIG. 10A depicts the results of a test in which glucose-stimulated insulin secretion was synchronized by a train of 5 carbachol (CCh) pulses.

FIG. 10B depicts the results of a control experiment.

FIG. 11A depicts the results of a control experiment performed with 5 human islets.

FIG. 11B depicts the results of a control experiment performed with 5 human islets.

FIG. 11C depicts the results of a control experiment performed with 5 human islets.

### DETAILED DESCRIPTION

The assays provided herein may be implemented on a glass microfluidic device, which may permit insulin secretion and its measurement to be coupled into a continuous flow channel, thereby allowing near real time detailing of rapid insulin secretion dynamics. The measurement may be made spectroscopically in a fluorescence anisotropy-based competitive assay, which may be based on the difference in molecular rotation between bound and unbound fluorescence reporters.

In some embodiments, the methods provided herein provide a 45% boost in signal-to-noise for fluorescence signal detection, 45% enhanced dynamic range, and/or a temporal resolution thrice improved. The methods provided herein may facilitate the measurement of dynamic insulin from single or grouped islets.

In some embodiments, the methods are sensitive and robust enough to detect or monitor insulin release from single mouse islets as well as human islets, and may do so at a fraction of the cost of typical insulin of ELISA kit-based experiments.

In some embodiments, the methods provided herein include a fluorescence anisotropy competitive immunoassay for online insulin detection from single and grouped islets using a piezoelectric pressure-driven fluid delivery system and a squaraine rotaxane fluorophore, SeTau-647, as the fluorescent label for insulin. In some embodiments, the fluidic system and microfluidic design produce a 36±1 s temporal resolution and flow rates <0.1% RSD.

Due at least to a longer lifetime and higher brightness compared to the previously used Cy5 fluorophore, SeTau-647 may increase the insulin quantification range by 45% and/or result in enhanced S/N of the measurements. In some embodiments, an all-optical system was suitable for measuring glucose-stimulated insulin secretion from single and groups of murine and human islets. Distinct islet entrainment of groups of murine islets by pulses of CCh was also observed in some embodiments, providing further evidence that pulsatile output from the ganglia can synchronize islet behavior.

The homogeneous assays provided herein may be widely used for examining, not only insulin secretion, but other secreted factors from different tissues.

### Systems and Devices

Systems and devices are provided herein for detecting or monitoring a target, such as insulin. A target is “detected” when its presence or concentration is determined at a particular time, and a target is “monitored” when it is “detected” continuously or at more than one particular time.

The devices and systems provided herein may include a microfluidic device. The microfluidic device may be formed of any suitable material, such as glass. In some embodiments, the microfluidic devices include an islet chamber. The islet chamber may include a feature, such as a reservoir, in which a sample may be disposed.

In some embodiments, the microfluidic devices herein include at least three fluidic inlets, wherein at least one of the fluidic inlets is upstream of an islet chamber, and at least one of the fluidic inlets is downstream of an islet chamber.

A fluidic inlet is “upstream of an islet chamber” when a fluid disposed in the fluidic inlet travels via one or more channels to the islet chamber when the microfluidic device is used as intended. A fluidic inlet is “downstream of an islet chamber” when a fluid disposed in the fluidic inlet does not travel to the islet chamber when the microfluidic device is used as intended.

In some embodiments, the microfluidic devices include (i) a first fluidic inlet upstream of the islet chamber, (ii) a second fluidic inlet upstream of the islet chamber, (iii) a third fluidic inlet downstream of the islet chamber, and (iv) a fourth fluidic inlet downstream of the islet chamber.

In some embodiments, the microfluidic devices include a first fluidic inlet in fluid communication with a first channel, and a second fluidic inlet in communication with a second channel. The first channel and the second channel may connect at one or more locations to a first main channel that includes an islet chamber.

In some embodiments, the microfluidic devices include a third fluid inlet in fluid communication with a third channel, and a fourth fluidic inlet in fluid communication with a fourth channel. The third channel and the fourth channel may connect with the first main channel at one or more locations downstream of the islet chamber to form a second main channel. Therefore, the second main channel may host a stream that includes the contents of the main channel, the third channel, and the fourth channel.

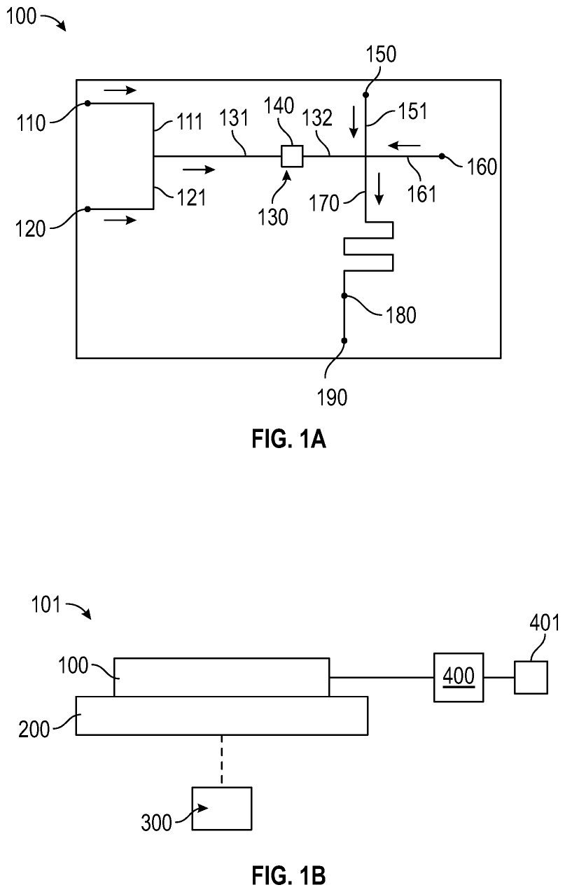

An embodiment of a microfluidic device is depicted at FIG. 1A. The microfluidic device 100 of FIG. 1A includes a first fluidic inlet 110 upstream of the islet chamber 140, and the first fluidic inlet 110 is in fluid communication with a first channel 111. The microfluidic device 100 also includes a second fluidic inlet 120 upstream of the islet chamber 140. The second fluidic inlet 120 is in fluid communication with a second channel 121. The first channel 111 and the second channel 121 connect at a single location to a first main channel having a first portion 131 upstream of the islet chamber 140, and a second portion 132 downstream of the islet chamber 140. The microfluidic device 100 also includes a third fluidic inlet 150 downstream of the islet chamber 140. The third fluid inlet 150 is in fluid communication with a third channel 151. The microfluidic device 100 also includes a fourth fluidic inlet 160 downstream of the islet chamber 140. The fourth fluidic inlet 160 is in fluid communication with a fourth channel 161, and the third channel 151 and the fourth channel 161 connect at a single location to the second portion 132 of the main channel to form a second main channel 170. The microfluidic device 100 also includes an outlet 190, and a detection point 180.

The channels of a microfluidic device may include microchannels. The channels may have any suitable dimensions. In some embodiments, the microfluidic devices include one or more microfluidic channels having a depth of about 60 μm to about 120 μm, a depth of about 70 μm to about 110 μm, a depth of about 80 μm to about 100 μm, or a depth of about 85 μm to about 95 μm.

In some embodiments, the microfluidic devices include one or more microfluidic channels having a width of about 150 μm to about 250 μm, of about 180 μm to about 220 μm, or about 190 μm to about 210 μm.

In some embodiments, the systems provided herein include a metal plate on which a microfluidic device is arranged. The metal place may define one or more voids configured to permit optical detection of a stream within a channel of the microfluidic device.

In some embodiments, the systems include a first fluid reservoir. The first fluid reservoir may be (i) pressurized with a first flow controller, (ii) in fluid communication with a fluidic inlet, such as a fourth fluidic inlet, or (iii) a combination thereof. Any of the fluids or components used in the methods described herein may be disposed in the first fluid reservoir, and the first fluid reservoir may be in fluid communication with any fluidic inlet of a microfluidic device. In some embodiments, a labeled target is disposed in the first fluid reservoir. The labeled target, as described herein, may be labeled with a squaraine rotaxane fluorophore.

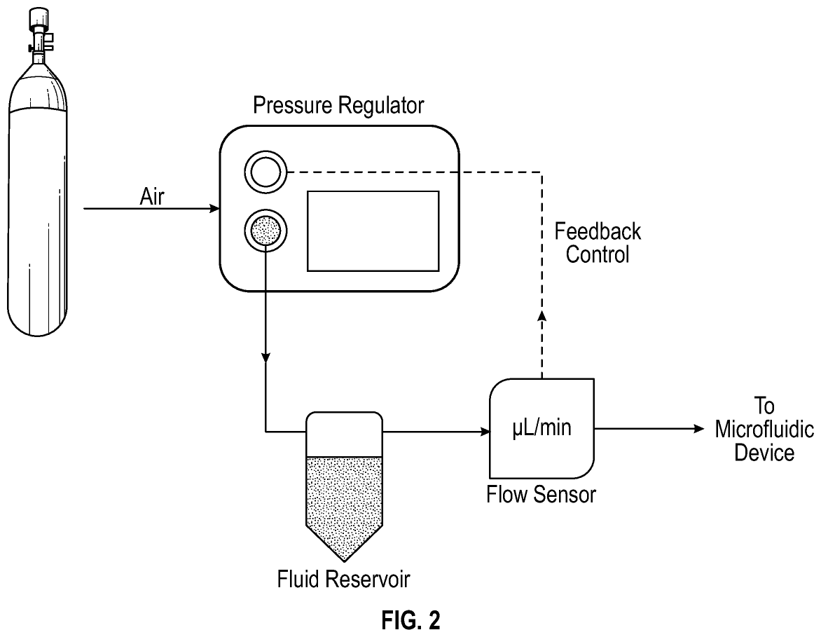

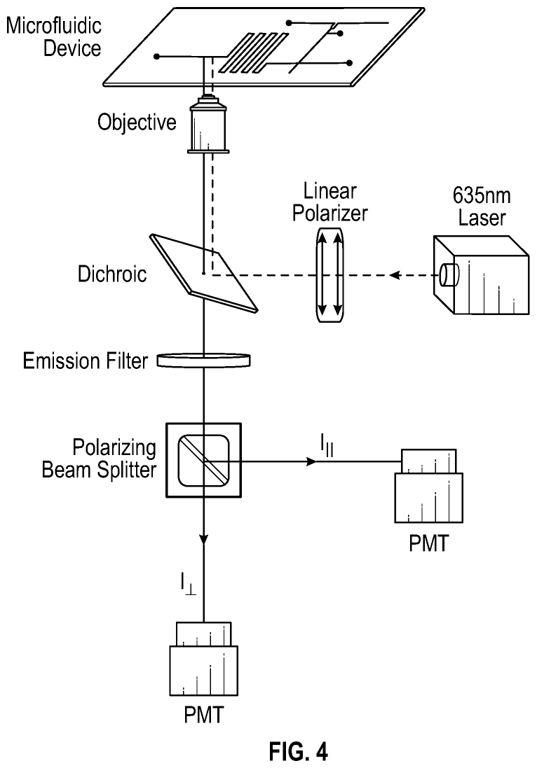

An embodiment of a system 101 for detecting or monitoring a target is depicted at FIG. 1B. The system 101 includes the microfluidic device 100 of FIG. 1A, and a metal plate 200 on which the microfluidic device 100 is arranged. The metal plate 200 defines a void (not shown) that aligns with the detection point 180 of the microfluidic device 100. The void (not shown) permits optical detection of a stream within the second main channel 170 of the microfluidic device 100 with the optical apparatus 300. The optical apparatus 300, in some embodiments, is the apparatus depicted at FIG. 4. The system 101 also includes a first fluid reservoir 400 pressurized with a first flow controller 401. The first fluid reservoir 400 is in fluid communication with the fourth fluidic inlet 160 of the microfluidic device 100. The first fluid reservoir 400 and first flow controller 401 may include those depicted at FIG. 2.

In some embodiments, the systems include a second fluid reservoir pressurized with a second flow controller, optionally a third fluid reservoir pressurized with a third flow controller, and optionally a fourth fluid reservoir pressurized with a fourth flow controller, wherein the second, third, and fourth fluid reservoirs are in fluid communication with different fluidic inlets of a microfluidic device, such as the first, second, and third fluidic inlets. In some embodiments, at least one of the flow controllers is a piezoelectric flow controller.

In some embodiments, the systems provided herein also include an optical apparatus. The optical apparatus may be configured to perform an analytical technique, such as a fluorescence anisotropy-based competitive assay. In some embodiments, the optical apparatus includes a laser, a linear polarizer, a dichroic mirror, an emission filter, a polarizing beam splitter, a photomultiplier tube, or a combination thereof

### Insulin Label

Any suitable label, such as a dye, may be used to label insulin as a reporter (e.g., fluorescence reporter) for the methods provided herein. For example, a dye may be selected that provides relatively high assay dynamic range, signal-to-noise ratio, or a combination thereof. In some embodiments, the dye has one or more favorable photophysical properties, such as a relatively large Stokes shift (e.g., at least 45 nm (e.g., 46 nm)), a relatively larger molar extinction coefficient (e.g., at least 200,000 M⁻¹ cm⁻¹), a relatively larger quantum yield (e.g., about 0.65), a relatively longer fluorescence lifetime (e.g., 3.2 ns) in aqueous solutions, or a combination thereof.

In some embodiments, the dye is a squaraine rotaxane fluorescence dye. In some embodiments, the dye is SeTau-647 (SETA BioMedicals, USA). In some embodiments, the use of SeTau-647 improves the dynamic range of the immunoassay and/or increases the reproducibility of measurements.

### Methods and Systems

Methods and systems are provided that may be used for detecting or monitoring a target, such as insulin. The methods, for example, may be used for the online detection or monitoring of insulin.

In some embodiments, the methods include providing a sample that includes an islet. The providing of the sample may include providing a microfluidic device that includes an islet chamber in which the sample is disposed.

In some embodiments, the sample includes at least one human islet. In some embodiments, the sample includes at least three human islets.

In some embodiments, the methods include contacting a sample with (i) glucose or (ii) glucose and a balanced salt solution to produce a first stream that includes a target insulin. The first stream, in some embodiments, is a perfusion flow from an islet chamber of a microfluidic device. A perfusion flow from the islet chamber may have a flow rate of about 0.1 to about 1 microliters/minute, about 0.2 to about 0.8 microliters/minute, or about 0.2 to about 0.5 microliters/minute.

In some embodiments, the contacting of the sample with glucose includes disposing glucose in at least one of a first fluidic inlet and a second fluidic inlet to deliver glucose to an islet chamber. In some embodiments, the contacting of the sample with glucose and a balanced salt solution includes disposing glucose and the balance salt solution in a first fluidic inlet and a second fluidic inlet, respectively, to deliver glucose and the balanced salt solution to an islet chamber.

In some embodiments, the methods also include disposing carbachol in at least one of the first fluidic inlet and the second fluidic inlet.

In some embodiments, the methods include contacting a first stream that includes a target insulin with an anti-insulin antibody and a labeled insulin to produce a second stream. The second stream may include the target insulin, an amount of an antibody-bound (B) labeled insulin, and an amount of free (F) labeled insulin. In some embodiments, the contacting of the first stream, which may be a perfusion flow from the islet chamber, with the anti-insulin antibody and the labeled insulin includes disposing the anti-insulin antibody and the labeled insulin in a third fluidic inlet and a fourth fluidic inlet of a microfluidic device, such as those described herein.

In some embodiments, the methods include determining a ratio of the amount of the antibody-bound (B) labeled insulin to the amount of the free (F) labeled insulin in the second stream. The ratio of B:F may be inversely related to a concentration of the target insulin in the second stream.

The ratio of B:F may be determined using any analytical method. In some embodiments, the ratio of B:F is determined homogenously. In some embodiments, the ratio of B:F is determined homogeneously using fluorescence anisotropy.

In some embodiments, the microfluidic device is arranged on a metal plate defining one or more voids configured to permit optical detection within a channel of the microfluidic device, and the methods include controlling a temperature of the metal plate with one or more heaters, heat sinks, or a combination thereof.

In some embodiments, the methods also include contacting an amount of insulin with an amount of a squaraine rotaxane fluorophore to form a labeled insulin. A weight ratio of the amount of insulin to the amount of the squaraine rotaxane fluorophore may be about 1:1 to about 1.5:1.

In some embodiments, the methods provided herein include performing a fluorescence anisotropy-based homogeneous assay of a liquid that includes a labeled target, wherein the labeled target is labeled with a squaraine rotaxane fluorophore.

In some embodiments, a fluid reservoir pressurized with a flow controller is in fluid communication with the first, second, third, and/or fourth fluidic inlet. The flow controller may be a piezoelectric flow controller. The fluid reservoir may be pressurized by a piezoelectric pressure regulator.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the descriptions provided herein, the terms “includes,” “is,” “containing,” “having,” and “comprises” are used in an open-ended fashion, and thus should be interpreted to mean “including, but not limited to.” When devices, systems, or methods are claimed or described in terms of “comprising” various steps or components, the devices, systems, or methods can also “consist essentially of” or “consist of” the various steps or components, unless stated otherwise.

The terms “a,” “an,” and “the” are intended to include plural alternatives, e.g., at least one. For instance, the disclosure of “a microfluidic device”, “a labeled insulin”, and the like, is meant to encompass one, or mixtures or combinations of more than one microfluidic device, labeled insulin, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, that microchannels have a depth of about 85 μm to about 95 μm. This range should be interpreted as encompassing about 85 μm and about 95 μm, and further encompasses “about” each of 86 μm, 87 μm, 88 μm, 89 μm, 90 μm, 91 μm, 92 μm, 93 μm, and 94 μm, including any ranges and sub-ranges between any of these values.

As used herein, the term “about” means plus or minus 10% of the numerical value of the number with which it is being used.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

The following reactions and equipment were in the following examples. All reagents for microfluidic assays and isolation and culture of islets were obtained from Sigma-Aldrich (Saint. Louis, Mo.) unless otherwise stated. NaOH, KCl, Tween-20, KCl, and HF were from EMD Chemicals (San Diego, Calif.). Glucose (dextrose), RPMI 1640, and gentamicin sulfate were from Thermo Fisher Scientific (Waltham, Mass.). Collagenase P (from Clostrdium histolyticum) was acquired from Roche Diagnostics (Indianapolis, Ind.). Cosmic Calf Serum was acquired from GE Healthcare Bio-Sciences (Pittsburgh, Pa.). Monoclonal Ab_{Ins} was purchased from Meridian Life Science, Inc. (Saco, Me.). All solutions were made with Milli-Q (Millipore, Bedford, Mass., USA) 18 MΩ·cm ultrapure water and filtered using 0.2 μm nylon syringe filters (Pall Corporation, Port Washington, N.Y.). Immunoassay reagents (Ins* and Ab_{Ins}) were prepared in TEAT-40 composed of 25 mM Tricine, 40 mM NaCl, 1 mM EDTA at pH 7.4 with an additional 0.1% Tween-20 (w/v) and 1 mg mL⁻¹ BSA. A balanced salt solution (BSS) made to pH 7.4 was used for preparing Ins standards and for islet perfusion. BSS was composed of 125 mM NaCl, 2.4 mM CaCl₂), 1.2 mM MgCl₂, 5.9 mM KCl, 25 mM HEPES, 1 mg mL⁻¹ BSA, and 3, 11 or 12 mM glucose. Reagents for isolation and culture of murine islets were prepared using known techniques.

### Example 1—Insulin Labeling and Purification of SeTau-647-Labeled Insulin

The protocol for labeling insulin (Ins) with SeTau-647-NHS (SETA BioMedicals, Urbana, Ill.) and purifying labeled insulin (Ins*) was adapted from methods used for Cy-5-labeled Ins (Lomasney, A. R. et al. Anal. Chem. 2013, 85 (16), 7919-7925). In this example, 1 mg of bovine insulin in 0.1 M NaHCO₃ (pH 9.3) was added to a vial containing 1 mg of SeTau-647. The mixture was incubated in the dark at room temperature for 30 minutes with gentle stirring every 5 minutes. The mixture was then separated using a PD-10 desalting column (GE Healthcare Bio-Sciences, Pittsburgh, Pa.). The first eluting band (SeTau-647-insulin) was collected and purified.

Further purification of SeTau-647-insulin was performed by liquid chromatography using a Beckman 127S solvent module pump and a Beckman 166 UV detector (Beckman Coulter, Indianapolis, Ind., USA). The column was a 25 cm×4.6 mm i.d. Symmetry300™ C4 (Waters Corp, Milford, Mass., USA) with a particle diameter of 5 μm. Separation was performed using 70% water containing 0.1% trifluoracetic acid (TFA):30% acetonitrile with 0.1% TFA with UV-Vis detection at 280 nm. Injections of 200 μL were made onto the column and all detected peaks were collected.

The fractions from multiple runs were pooled and concentrated to dryness (Savant Speedvac, Thermo Fisher Scientific, Waltham, Mass.). Each pooled fraction was checked for binding to Ab_{Ins} by adding enough Ab_{Ins} in TEAT40 to produce a 24 nM final concentration. The resulting mixture was separated by capillary electrophoresis (Beckman-Coulter PA800, Brea, Calif.) with a 25 μm i.d.×60 cm fused silica capillary (Polymicro Technologies, Phoenix, Ariz.) using a 20 kV separation potential. Detection was accomplished using laser-induced fluorescence with a 635 nm laser (AixiZ, Houston, Tex.) and a 663 nm longpass filter. Separation buffer contained 150 mM Tricine and 20 mM NaCl made to pH 7.4. After determining which fraction of Ins* bound to Ab, the Ins* was quantified by UV-VIS spectroscopy using the molar extinction coefficient of SeTau-647 (ϵ=200,000 M⁻¹ cm⁻¹ at 649 nm). Labeled insulin was aliquoted and stored in the dark at −80° C. Each aliquot contained 2.5 μL of 20 μM Ins* which was then diluted to 45 nM in TEAT-40 for microfluidic experiments.

### Example 2—Microfluidic Device and System

The microfluidic device of this example was fabricated in borosilicate glass using photolithography and wet etching techniques as previously detailed (see, e.g., Lomasney, A. R. et al., Anal. Chem. 2013, 85 (16), 7919-7925; and Bandak, B. et al. Lab Chip 2018, 18 (18), 2873-2882). Microfluidic channels were 90×195 μm (depth×width at middle) as measured by an SJ-410 surface profiler (Mitutoyo Corp., Aurora, Ill.).

Fluidic inputs and the 120 nL islet chamber were drilled using 0.02″ and 0.012″ diamond-tipped drill bits, respectively (Wolfco Inc., Bozrah, Conn.).

The device had 4 fluidic inlets: the 2 upstream of the islet chamber delivered BSS with different concentrations of secretagogues while the 2 inlets downstream of the islet chamber delivered 45 nM Ins* and 90 nM Ab_{Ins}, both in TEAT40.

Each inlet was connected to a fluid reservoir pressurized by a piezoelectric flow controller (Elvesys, Paris, France) with their flow rates regulated by in-line flow sensors (Elvesys, Paris, France). The error associated with these sensors were specified at 2% RSD by the manufacturer and were periodically verified. The final Ins* and AbIns concentrations were determined by the ratio of flow rates from each of these inlets to the total flow rate. Throughout the remainder of the examples, the final, fully mixed concentrations were used.

Temperature Control:

To ensure physiological temperature within the islet chamber, the entire microfluidic device was secured in the center of a custom-built copper plate with 2 mm wide slits cut in the shape of the channels to allow for optical detection within the device. On each corner of the plate, a thermoelectric (Peltier) heater (TEC1-12706, Hebeit I. T., Shanghai, China) was attached using a thermal adhesive paste (Arctic Silver Inc., Visalia, Calif.). On the opposite side of each Peltier was attached a 40×40×10 mm (L×W×D) aluminum heat sink. Each Peltier was powered by an 18 V, 3 A power supply (Extech Instruments, Nashua, N.Y.). For temperature measurement, a calibrated T-240C micro-thermocouple (Physitemp Instrument, Inc. Clifton, N.J.) connected to a TAC80B-T thermocouple-to-analog converter (Omega Engineering, Inc., Stamford, Conn.) was arranged between the copper plate and microfluidic device. The temperature was input into a LabView program (National Instruments, Austin, Tex.) written in-house via an NI-PCIe 6321 data acquisition (DAQ) card (National Instruments). The program, through the DAQ card and a Crydom CMX60D10 relay, interrupted the power supply to the Peltier circuit to maintain the desired temperature. Islet chamber temperature throughout a typical 60 minute experiment was measured to be 37.0±0.3° C.

Microfluidic Device and Perfusion System Characterization:

In some tests, gravity-driven flow was used to deliver reagents to the microfluidic chip. Although there are several advantages to this passive pressure-driven mechanism of fluid delivery, one major drawback was its inability to automatically correct flow rate fluctuations when bubbles or clogs developed in the fluidic lines or microfluidic device. To improve the robustness of flow, a variable-pressure system was implemented in this example.

A schematic of the microfluidic system used in this example is presented at FIG. 2. In the system depicted at FIG. 2, a piezoelectric pressure regulator was used to deliver reagents from fluid reservoirs through an inline flow sensor to the microfluidic device. Flow rates were monitored by the regulator via a feedback control loop.

Fluidic reservoirs containing assay reagents were pressurized with filtered air by a piezoelectric pressure regulator. As liquid flowed from the reservoirs into the microfluidic device, flow rates were continuously monitored by an in-line flow sensor connected in a feedback loop to the piezoelectric regulator which adjusted the pressure output to maintain flow stability.

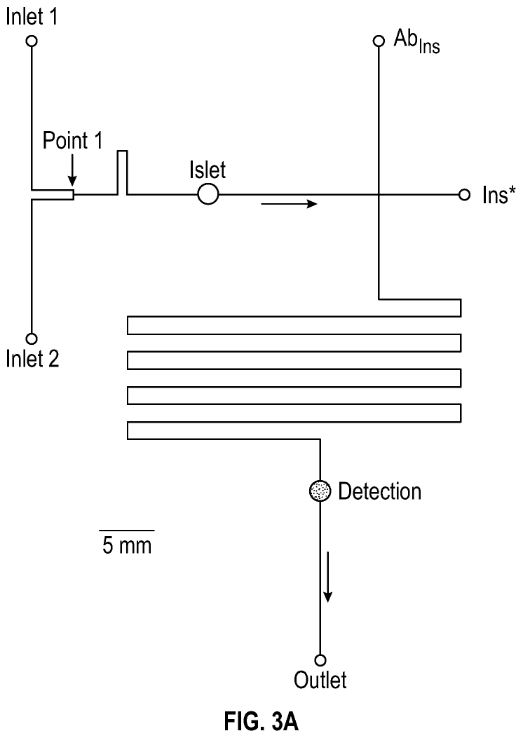

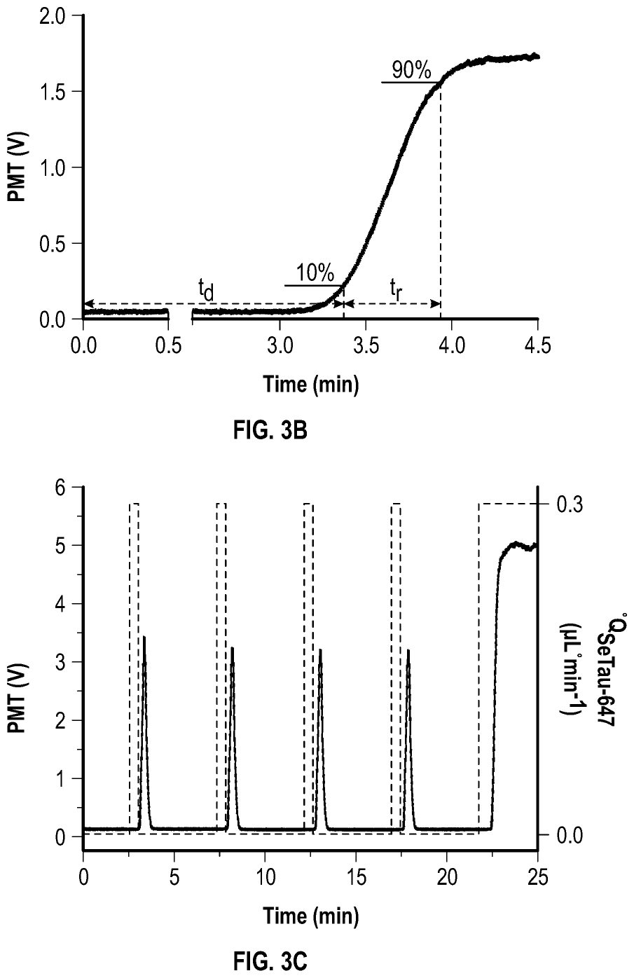

FIG. 3A depicts a schematic of the embodiment of the microfluidic chip used in this example. In FIG. 3A, the 2 fluidic inlets upstream of the islet chamber (dot under “Islet”) are indicated. The 2 remaining inlets are designated for Ab_{Ins} and Ins*, respectively, for islet and insulin calibration experiments. Immunoassays were equilibrated in the 160 mm long mixing channel before fluorescence measurement at the detection point shown (dot adjacent “Detection”). The arrows of FIG. 3A indicate the direction of flow.

For all experiments in the following examples, the perfusion flow rate through the islet chamber was maintained at 0.3 μL min⁻¹, but other rates are envisioned. The Ins* and Ab_{Ins} flow rates were set at 0.4 and 0.8 μL min⁻¹, respectively, for a total flow rate at the detection point of 1.5 μL min⁻¹.

### Example 3—Optical Detection System for Microfluidic Device

In this example, a 635 nm laser (Coherent Inc. Santa Clara, Calif.) was used as the excitation source. The laser power was reduced from 25 mW to 7.8 mW with a neutral density filter (Thorlabs inc., Newton, N.J.). An achromatic fiberport collimator (PAF2S-7A, Thorlabs) coupled the attenuated beam into a multimode fiber optic bundle (Ceramoptec, Sunnyvale, Calif.) which then fed into a telescoping lens tube (SM1NR1, Thorlabs).

Contained in the lens tube was an achromatic doublet (AC254-080-A-ML, Thorlabs) to collimate the excitation beam followed by a quartz-wedge achromatic depolarizer (DPU-25-A, Thorlabs) to randomize the optical polarization of the beam. The beam entered the back of an Eclipse TS-100 microscope (Nikon Instruments Inc., Melville, N.Y.), was polarized in the desired orientation using a linear polarizer (WP25M-VIS, Thorlabs), and reflected by a dichroic mirror (XF2035, Omega Optical Inc., Brattleboro, Vt.). The reflected light was then focused by a 40×, 0.6 NA objective (Nikon) into the microfluidic channel at the detection point. Fluorescence emission was collected with the same objective, transmitted through the dichroic, and directed into a 2-channel microscope photometer (Horiba Scientific, Piscataway, N.J.). Within the photometer, the light passed through a spatial filter, a 665 nm long pass filter (HQ665LP, Chroma Technology Corp., Bellows Falls, Vt.), and a 635 nm notch filter (ZET635NF, Chroma Technology Corp.).

The emission beam was then split by a polarizing beam splitter cube (PBS101, Thorlabs) into its parallel and perpendicular polarized components (with respect to the excitation polarization). Each polarized component passed through a complementary linear polarizer prior to impinging on separate photomultiplier tubes (PMTs) (R10699, Hamamatsu Photonics, Middlesex, N.J.). Because most of the fluorescence emission collected by the objective was parallel to the polarization axis of the excitation beam, the gain of the PMT for detection of the perpendicular component was set to 0.1 μA V⁻¹ whereas the gain for the PMT used for detection of the parallel component was 1.0 μA V⁻¹. Data from both PMTs were collected at 1000 Hz with a DAQ card (National Instruments, USB 6009) using a LabView program.

The microfluidic system was held on the stage of a microscope, and the optical system depicted at FIG. 4 was used to evaluate the temporal resolution of the system. In the optical setup of FIG. 4, linearly polarized 635 nm fluorescence excitation was reflected by a dichroic and focused into the microfluidic channel. Laser-induced fluorescence was collected by the objective, transmitted through the dichroic and the emission filters, and the parallel and perpendicular components of laser-induced fluorescence were measured by PMTs.

Reservoirs connected to inlets 1 and 2 contained BSS and 20 nM SeTau-647, respectively. BSS was delivered to the 2 remaining inlets (Ab_{Ins} and Ins* in FIG. 3A) at 0.4 and 0.8 μL min⁻¹ with the islet chamber sealed. Initially, BSS flowed into the device through inlet 1 at 0.3 μL min⁻¹ while the flow of SeTau-647 from inlet 2 was set to 0.0 μL min⁻¹. The flow rates from inlets 1 and 2 were then reversed so that SeTau-647 was delivered and fluorescence was measured at the detection point of the device.

As shown at FIG. 3B, after a delay time (t_{d}), the fluorescence increased until the signal plateaued. FIG. 3B depicts the results of the flow characterization experiment, and depicts the change in fluorescence signal when SeTau-647 flow was initiated from point 1 at 0 min. Signal was measured at the detection point of FIG. 3A. The x axis break at 0.5 min was included for convenience and clarity.

Because time 0 corresponded to the time at which SeTau-647 began to flow, t_{d} quantified the time for travel from point 1 in FIG. 3A to the detection point. The t_{d} was determined as the intensity to reach 10% of the final signal at the detection point, and was 3.2±0.2 min (n=4 trials). The response time (tᵣ) was the time required for a change in fluorescence signal from 10 to 90% of the final signal at the detection point. This value was 36±1 s (n=4), a 3-fold improvement on the previous gravity-driven perfusion system (Schrell, A. M. et al. Anal. Methods 2017, 9 (1), 38-45), and was taken as the temporal resolution of the device. The values provided in this example were for trials within a single microfluidic device. Although the numbers varied slightly more between devices, the intra-device variation was similar.

To evaluate how pulses of CCh would attenuate when delivered to the islet chamber, a pulse characterization experiment was performed by measuring sequential pulses of SeTau-647 at the islet reservoir. Inlets 1 and 2 contained BSS and SeTau-647, respectively, and the total flow rate from these reservoirs to the islet chamber was held constant at 0.3 μL min⁻¹. A series of 30 second pulses was generated by repeatedly switching the flow between the two inlets, followed by a constant flow from the SeTau-647 inlet. The PMT voltage (FIG. 3C) showed the arrival of all 4 pulses as well as a plateau when constant dye was perfused. A 53±3% attenuation in signal was observed for the height of the pulses as compared to the constant dye delivery. The delay time for travel between point 1 and the islet chamber was measured to be 0.71±0.01 min (n=4) using the pulse profile shown at FIG. 3C. In FIG. 3C, the trace (right axis) showed the profile of programmed flow rates of SeTau-647 solution delivered into microfluidic device from inlet 2. The trace (left axis) was the fluorescence signal measured by a PMT as a function of time at the islet chamber during SeTau-647 pulsing. Longitudinal diffusion resulted in a 53±3% pulse attenuation at the islet chamber.

Assay Improvement with Squaraine Rotaxane Fluorophore:

Immunoassays using fluorescence anisotropy are typically competitive, whereby the target and a tagged analog of the target compete for binding sites to a limited amount of antibody. In the case of insulin measurements, insulin (Ins) released from islets competes with fluorescently-tagged insulin (Ins*) for a limiting amount of anti-insulin antibody (Ab_{Ins}). Quantification, in this example, included measuring the amount of antibody-bound (B) and free (F) tagged species (Ins*), with the ratio, B/F, inversely related to the concentration of target (Ins) in the mixture. In this example, this ratio was determined homogeneously using fluorescence anisotropy because each of these species has a unique anisotropy as seen by the Perrin equation:

$$r = \frac{{r}_{0}}{1 + τ / θ} \qquad 1$$

wherein r₀ is the fundamental anisotropy of the fluorophore, τ its fluorescence lifetime, and θ its rotational correlation time, defined as:

$$θ = \frac{η \overline{V}}{R T} \qquad 2$$

where η is viscosity, ∇ is the molar volume, R is the universal gas constant, and T is temperature. In the case of the competitive insulin immunoassay, the increased ∇ of the bound Ins*−Ab_{Ins} complex compared to free Ins* lead to a higher anisotropy for the bound species (r_{B}) than for the free Ins* (r_{F}). The immunoassay was performed homogeneously because the total anisotropy ( [symbol] r [symbol] ) of the mixture was a sum of r_{B} and r_{F} weighted by their fractional amounts:

$$〈 r 〉 = {f}_{B} {r}_{B} + {f}_{F} {r}_{F} \qquad 3$$

wherein fₛ and f_{F} are the fractional amount of B and F Ins*, respectively. Every insulin concentration resulted in a unique combination of f_{B} and f_{F}, and therefore, a distinct [symbol] r [symbol] . The competitive assay was suitable for this measurement technique due to the large differences in ∇ between the B and F Ins*. The [symbol] r [symbol] was determined experimentally by exciting the solution with linearly polarized light and measuring the degree of depolarization in the emission normalized to the total fluorescence emission:

$$〈 r 〉 = \frac{{I}_{II} - {I}_{⊥}}{{I}_{II} + 2 * {I}_{⊥}} \qquad 4$$

with l_{∥} and l_{⊥} representing the fluorescence emission intensities parallel and perpendicular, respectively, to the polarization of the excitation light. Equation 4 assumed equal detection sensitivity for the two polarization states.

The fluorophore used in this example, SeTau-647, is a member of a class of squaraine rotaxane fluorescent probes which has similar excitation and emission wavelength maxima to Cy5, but SeTau-647 has more favorable photophysical properties, including a larger Stokes shift (46 nm), a larger molar extinction coefficient (200,000 M⁻¹ cm⁻¹), a larger quantum yield (0.65), and a longer fluorescence lifetime (3.2 ns) in aqueous solutions. Encapsulation of the squaraine chromophore by the rotaxane macrocycle may provide protection of the dye thereby increasing chemical stability and photobleaching resistance.

A comparison of calculated immunoassay calibration curves was made by plotting the calculated anisotropy ( [symbol] r [symbol] _{calc.}) vs. insulin concentration ([Ins]) using Cy5-labeled Ins (Ins*_{Cy-5}) and SeTau-647-labeled Ins (Ins*_{SeTau-647}) as the Ins*. To perform these calculations, the r_{B} and r_{F} for Ins*_{Cy-5} and Ins*_{SeTau-647} were calculated using equations 1 and 2 with θ values for each species assuming 0 degrees of hydration and at 37° C. Values for τ and r₀ were obtained from the literature (Huang, Z. et al. J. Phys. Chem. A 2006, 110 (1), 45-50; Widengren, J. et al. J. Phys. Chem. A 2000, 104 (27), 6416-6428; Luschtinetz, F. et al. Bioconjug. Chem. 2009, 20 (3), 576-582; and Webster, S. et al. Chem. Phys. 2008, 348 (1-3), 143-151).

Various properties of the B and F species for both labels were used to calculate r_{B} and r_{F}. Once these two anisotropies were calculated, f_{B} and f_{F} at different unlabeled Ins concentrations were calculated using mass action and equilibrium equations. These values were then used to determine [symbol] r [symbol] _{calc.} using equation 3 (see below).

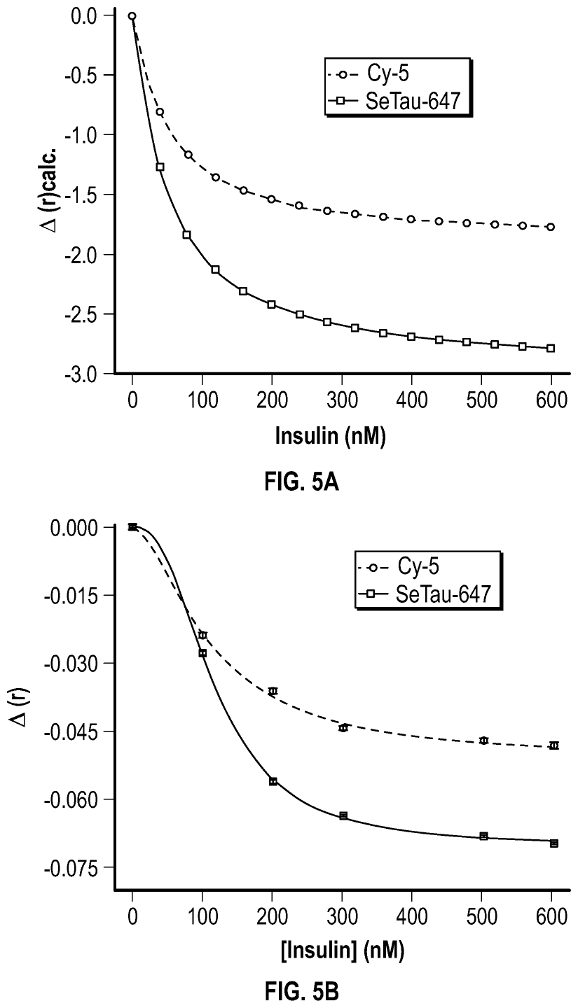

FIG. 5A shows the calculated immunoassay calibration curves for both fluorophores. The Ins calibration curves of Δ [symbol] r [symbol] _{calc.} vs. [Ins] from 0 to 600 nM using Ins*_{Cy-5} and Ins*_{SeTau-647} were computed using their photophysical properties and mathematically calculated B/F ratios as explained in the text. All data points were plotted relative to the anisotropy at 0 nM Ins and both curves show the sigmoidal decrease in anisotropy as a function of insulin, as expected for a competitive assay. To quantify assay improvement, the change in anisotropies (Δ [symbol] r [symbol] _{calc.}) for both curves from 0 to 600 nM Ins were compared. A 57% larger change was found with SeTau-647 compared to Cy5 due to the larger τ of the former. Conceptually, the larger value permits a higher degree of depolarization resulting in a greater change in anisotropy between r_{B} and r_{F} in equation 3 (see below).

Following these calculations, a comparison of experimentally-obtained calibration curves using Ins*_{Cy-5} and Ins*_{SeTau-647} was made (FIG. 5B). Online experimental calibration curves each of 0 to 600 nM [Ins] with 24 nM Ab_{Ins}, and 24 nM of either Ins*_{Cy-5} or Ins*_{SeTau-647}. Δ [symbol] r [symbol] values in A and B were taken as relative to the average measured anisotropy at 0 nM. SeTau-647 was shown to produce improved assay range from both theoretical A and experimental B results. Measurements were relative to the mean anisotropy at 0 nM insulin. A 45% increase in the range of anisotropy values was observed with Ins*_{SeTau-647}, similar to the calculated increase in anisotropy values. Additionally, due to the superior quantum yield of the squaraine rotaxane compared to Cy-5, the S/N of the measurements were enhanced, resulting in a 44% decrease in the average SD across the 6-point calibration curves. The LOD, limited in competitive assays by the equilibrium constant of the antibody-antigen reaction, was 20 nM for both curves since both used the same Ab_{Ins}.

Online Glucose Stimulated Insulin Secretion (GSIS) Measurements:

Initial islet experiments were performed with murine islets to compare insulin secretion profiles using this new system to previous reports (see, e.g, Shackman, J. G. et al. Lab Chip 2005, 5 (1), 56-63; Yi, L. et al. Anal. Chem. 2016, 88 (21), 10368-10373; Dishinger, J. F. et al. Anal. Chem. 2009, 81, 3119-3127; Yi, L. et al. Lab Chip 2015, 15 (3), 823-832; Lomasney, A. R. et al. Anal. Chem. 2013, 85 (16), 7919-7925; and Bandak, B. et al. Lab Chip 2018, 18 (18), 2873-2882). For single islet experiments, fluidic reservoirs leading to inlets 1 and 2 contained BSS with 3 and 11 mM glucose, respectively. After loading and sealing an islet in the microfluidic chamber, 3 mM glucose was applied for 5 minutes to condition the islet to flow. After this rinse, anisotropy measurements commenced with 3 mM glucose for 2 min, followed by 11 mM for 40 min, and then a return to 3 mM.

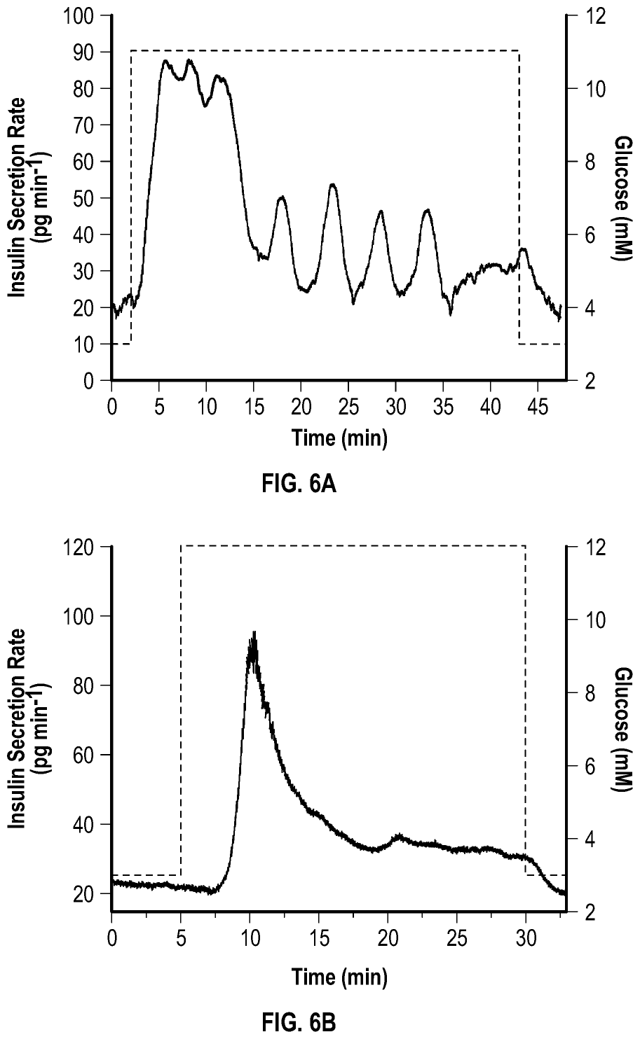

FIG. 6A shows a representative GSIS profile from a single murine islet. In both experiments, the trace (left y-axis) was the insulin secretion rate whereas the blue profile (right axis) was the glucose stimulus. In FIG. 6A, the islet was challenged with an 11 mM glucose challenge for 40 min during which biphasic, pulsatile insulin release was observed. The basal insulin release rate was 21±2 pg min⁻¹ measured over 2 minutes and the phase 1 secretion averaged over 7 minutes was 82±3 pg min⁻¹. After this burst, pulsatile insulin secretion, consistent with the foregoing previous reports, was observed with insulin oscillations peaking at 49±3 pg min⁻¹. Insulin returned to basal levels when the glucose challenge was removed.

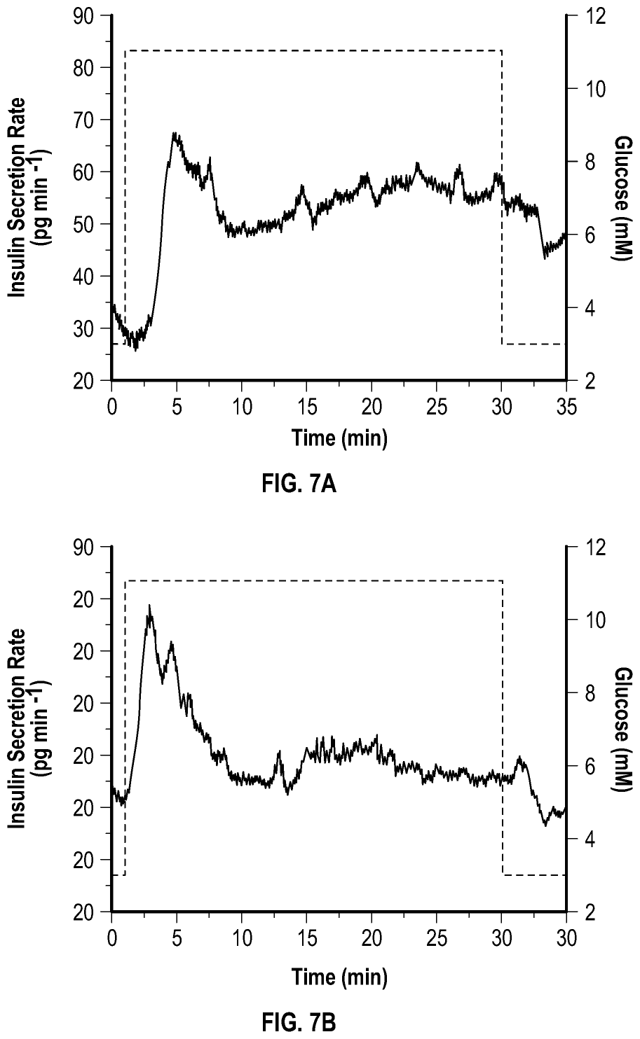

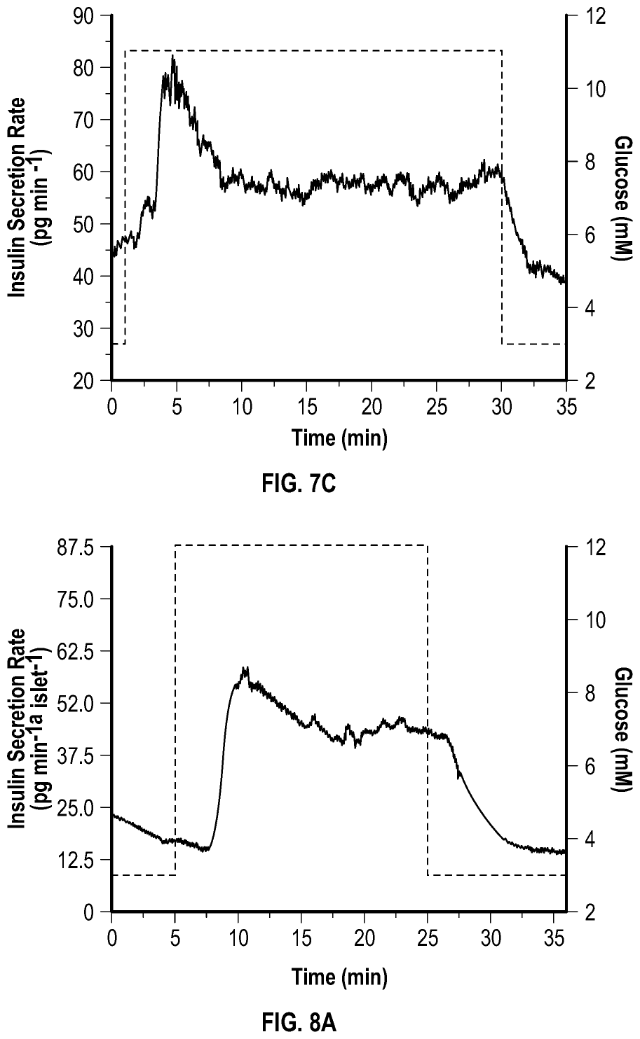

FIG. 7A, FIG. 7B, and FIG. 7C show the insulin secretion profiles of 3 additional murine islet GSIS experiments. The islet in each experiment was stimulated with 11 mM glucose for 30 minutes during which insulin release was measured. In each plot, the trace (left axis) was the rate of insulin secretion from the islet. The other profile (right axis) represented the glucose challenge. Similar trends were observed with biphasic insulin release from each islet, although phase 2 oscillations were not marked.

A representative example of GSIS monitoring from a group of 5 islets in the microfluidic chamber is shown at FIG. 6B. For this experiment, fluidic reservoirs connected to inlets 1 and 2 contained BSS with 3 and 12 mM glucose, respectively. The islets were rinsed with 3 mM glucose for 5 min showing a basal insulin release rate of 22±3 pg min⁻¹ islet⁻¹ for that duration. Glucose levels then rose to 12 mM during which a biphasic insulin response profile was observed. Phase 1 presented as a burst of high insulin release rate, peaking at 87±17 pg min⁻¹ islet⁻¹ for 1 minute whereas phase 2 showed a more sustained, but decreased, release rate of 34±7 pg min⁻¹ islet⁻¹ measured over 10 minutes. Secretion rates returned to basal levels when glucose was lowered to 3 mM.

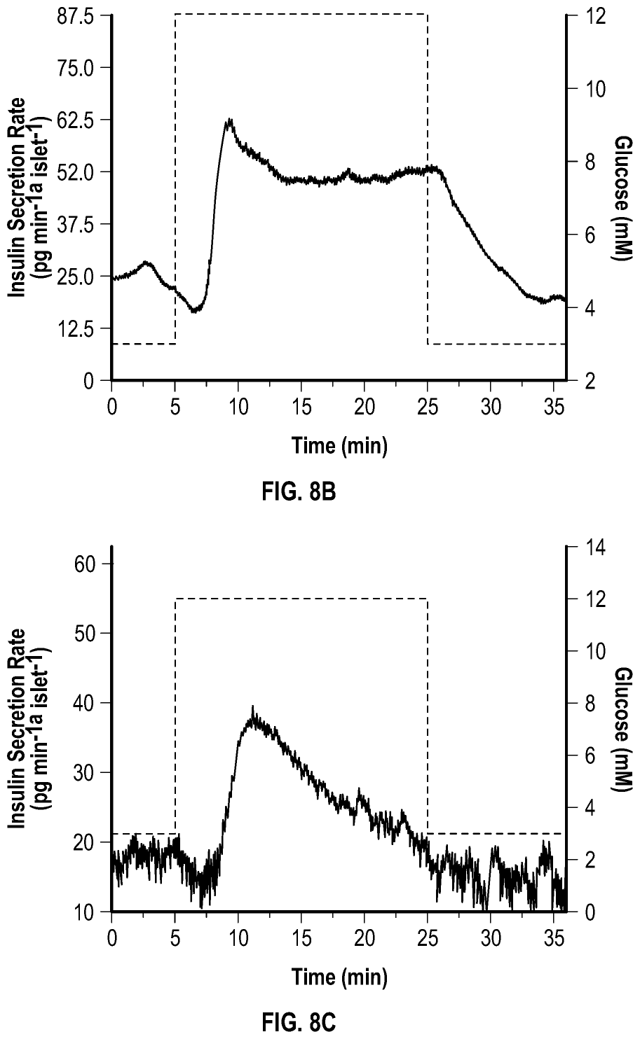

FIG. 8A, FIG. 8B, and FIG. 8C show 3 additional GSIS experiments. Insulin release rates were profiled with the trace (left axis). Each experiment consisted of 4 islets in the chamber. Islets in each experiment were stimulated with 12 mM glucose for 20 min (profile, right axis) during which insulin release was measured. In each experiment, 4 islets were loaded into the chamber and exposed to 12 mM glucose. Biphasic insulin release was observed during the glucose challenges, as expected. For both single and multiple islet experiments, the insulin secretion rates per islet are comparable with previously reported insulin secretion rates under similar glucose challenges.

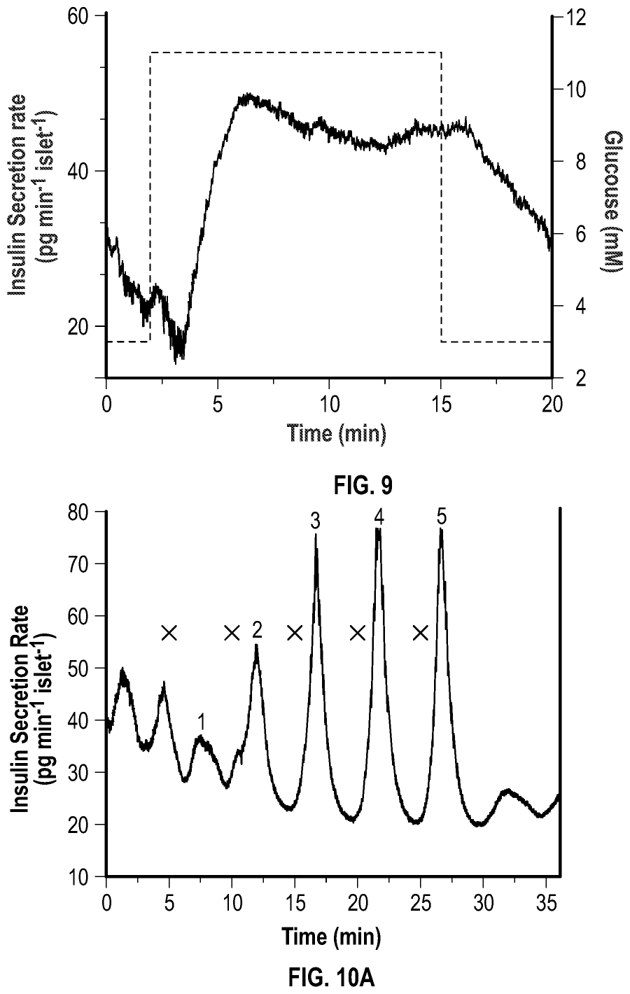

Due to the increased significance in understanding the biology of human disease, also tested was the ability to measure GSIS from human islets using this system. As a representative case (FIG. 9), 3 islets from a healthy female were placed in the chamber and insulin secretion recorded for 2 min with a 3 mM glucose perfusion. Insulin release rates during this period were 26±9 pg min⁻¹ islet⁻¹. Biphasic insulin release was then observed when 11 mM glucose was delivered to the batch. An insulin secretion rate of 49±1 pg min⁻¹ islet⁻¹ was measured during 30 seconds at the apex of phase 1 release and a relatively constant phase 2 average of 34 pg min⁻¹ islet⁻¹ from 12 to 16 minutes was observed. Insulin secretion did not readily return to baseline levels with 3 mM glucose. This secretion profile for human islets is consistent with what has been observed previously (see, e.g., Henquin, J.-C. et al. Diabetes 2006, 55, 3470-3477; Cabrera, O. et al. Cell Transplant. 2008, 16 (10), 1039-1048; Henquin, J.-C. et al. Am. J. Physiol. Endocrinol. Metab. 2015, 309, E640-E650; and Lenguito, G. et al. Lab Chip 2017, 17, 772-781).

The examples herein showed the capabilities of the homogeneous, online fluorescence anisotropy competitive immunoassay to quantify biphasic GSIS dynamics from both single and grouped murine and human islets. The improved assay range and higher S/N offered by SeTau-647, as well as the sub-minute temporal resolution of this microfluidic system allowed observations of insulin secretion dynamics that were unobservable in previous homogeneous assays.

Repetitive Activation of M3 Receptors Synchronize Murine Islets:

CCh, an M3R agonist, synchronizes glucose-induced islet activity as measured by synchronized oscillations of [Ca²⁺]ᵢ levels. Because no recording of insulin release has been reported after delivery of CCh pulses, synchronized hormone secretion had not previously been verified. Using the newly-developed anisotropy method, it was tested whether GSIS from grouped islets can be entrained during periodic application of CCh.

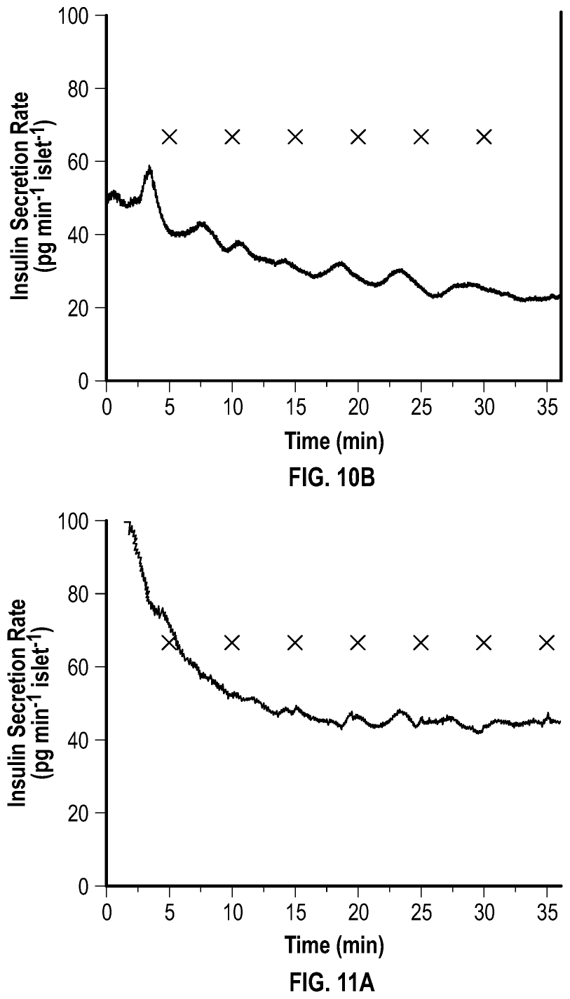

For this experiment, reagent reservoirs connected to inlets 1 and 2 contained 11 mM glucose and 10 μM CCh in 11 mM glucose, respectively. A group of 5 islets were loaded into the islet chamber that had been filled with 11 mM glucose. After sealing the chamber, islets were continuously perfused with constant 11 mM glucose from inlet 1. CCh pulses were generated by switching the flow from inlet 1 to inlet 2. Pulse profiles consisted of 5 pulses (denoted by ‘x’ in FIG. 10A and FIG. 10B) each delivered for 30 s every 5 min. Considering broadening of each pulse (FIG. 3C), islets were exposed to an effective [CCh] of 4.7 μM.

In FIG. 10A and FIG. 10B, the glucose level remained constant at 11 mM. In the islet chamber during each experiment were 5 islets. In FIG. 10A, GSIS was synchronized by a train of 5 CCh pulses. Each 30 second pulse, ‘x’, was delivered every 5 minutes from 5 to 25 minutes. Numbered insulin peaks highlight oscillations likely stimulated by a CCh pulse. After the final pulse, the distinct insulin oscillations ceased. FIG. 10B depicts the results of a representative control experiment, with flow switches denoted by the ‘x’. From the insulin secretion trace, as no CCh was present, GSIS was unsynchronized.

As shown in FIG. 10A, in the time prior to the first CCh pulse, only small insulin pulses were observed, which were likely due to the random overlap of insulin oscillations from the individual islets. Once the CCh pulses began, however, the measured insulin secretion profile was noticeably different. Whereas the first insulin spike after CCh delivery (labeled as peak 1 in FIG. 10A) was slightly dampened, all subsequent pulses of CCh facilitated significantly higher spikes of insulin, with the final 3 spikes averaging 78±12 pg min⁻¹ islet⁻¹. This result complemented previous work (see, e.g., Adablah, J. E. et al. PLoS One 2019, 14 (2)) that showed potentiation and 1:1 entrainment of islet activity by 5 minute periodic CCh pulses. These CCh-potentiated oscillations promptly declined after delivery of the final pulse at 25 min.

To ensure that synchronization was due only to the periodic application of CCh and not to any feature of the microfluidic system itself, control experiments were performed by performing a similar pulse profile, but without CCh present. This ensured that the islets remained perfused with only 11 mM glucose even while the pulse profile was being delivered. As shown in FIG. 10B, no discernible synchronization of insulin release was observed, only minor pulses similar to that observed prior to initiation of CCh pulses in the earlier experiment.

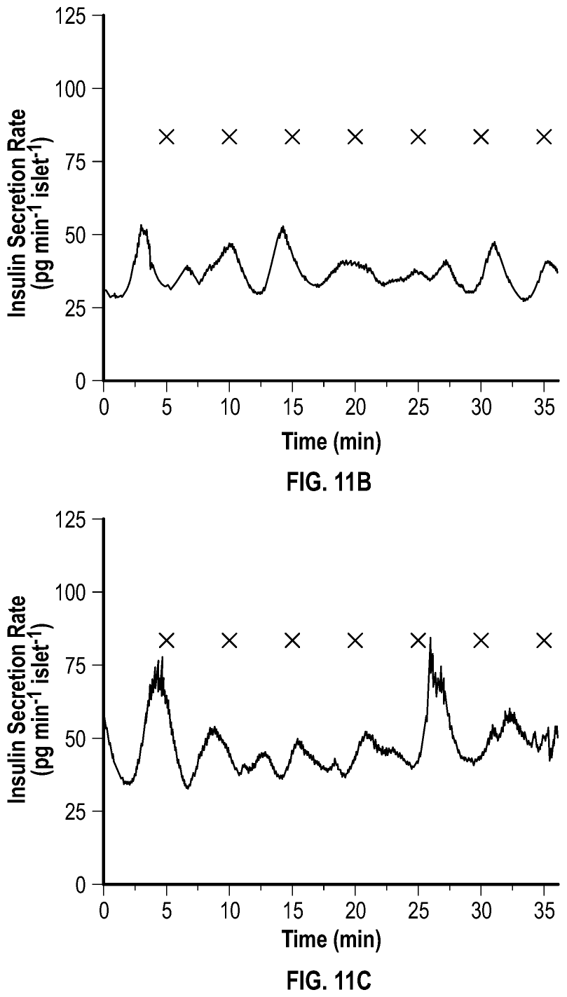

A total of 4 control experiments were performed in this way with the remaining 3 shown at FIG. 11A, FIG. 11B, and FIG. 11C. For these experiments, insulin secretion was unsynchronized without CCh. Three control experiments were performed with 5 islets in FIG. 11A, FIG. 11B, and FIG. 11C. The ‘x’ in each plot is a pulse of the fluidic system (without CCh). The tail end of the phase 1 burst was visible in the early stages of insulin measurement in FIG. 11A. Oscillations observed in FIG. 11B and FIG. 11B were short-lived and irregular, and showed no dependence to the glucose pulses applied.

The method of the foregoing examples demonstrated significant improvements for allowing the online fluorescence anisotropy competitive insulin immunoassay to quantify biphasic GSIS dynamics from single and grouped pancreatic islets and for continued exploration of the hypothesis of ganglia-induced islet synchronization. The overall design of the microfluidic system produced sub-minute temporal resolution for insulin measurements, low enough to observe the fast oscillation dynamics of single islets. The system performed well due, at least in part, to the use of SeTau-647 with its longer fluorescence lifetime than Cy-5. This resulted in increased fluorescence emission depolarization and a greater anisotropy shift between low and high antigen concentrations. To demonstrate the applicability of the system, online dynamic insulin release from murine and human islets after glucose induction were measured. Synchronized GSIS from a group of islets stimulated with pulses of CCh were observed, further supporting the hypothesis of ganglia-induced islet synchronization. The online homogeneous competitive immunoassay could be applicable as a user-friendly and affordable approach for the quantification of biologically relevant targets other than insulin with slight changes to the immunoassay reagents.

### Example 4—Procurement and Culture of Islets

Islets from 2 mice were pooled, incubated in RPMI 1640 containing 11 mM glucose, L-glutamine, 10% Cosmic Calf Serum, 100 U mL⁻¹ penicillin, 100 μg mL⁻¹ streptomycin, and 10 μg mL⁻¹ gentamycin at 37° C. and 5% CO₂. Islets were used no more than 4 days after isolation. Prior to each experiment, islets were rinsed in BSS containing 3 mM glucose for at least 5 minutes before loading into the islet chamber by sedimentation with a pipette. After loading, the islet chamber was sealed with a piece of adhesive PCR film.

Human islets, procured from Prodo Laboratories (Aliso Viejo, Calif.), were obtained from a deidentified cadaveric organ donor and, therefore, were exempt from Institutional Review Board approval. The donor was a 40 year old Hispanic female, 67″, 203 lbs., 31.8 BMI, 4.8% HbA1c, and without a history of diabetes. Incubation of human islets was performed at 37° C. and 5% CO₂ in PIM(S) islet-specific media (Prodo Labs).

### Example 5—Data Analysis

Data collected from both PMTs during experiments were converted to anisotropy using equation 4. Anisotropy traces were smoothed by a 1000-point moving boxcar average. To quantify Ins, online calibration curves were performed using 24 nM Ins* and Ab_{Ins} and 0 to 400 nM Ins for experiments with multiple islets. For single islets experiments, the Ins concentrations were halved and immunoassay reagent concentrations were reduced to 18 nM. Calibration curves were generated after islet experiments and were performed at 37° C. Each point on a curve is the average anisotropy after a 1 min collection with error bars representing ±1 standard deviation (SD). Calibration points were fitted to a 4-parameter logistic curve. The equation from the curve was used to convert anisotropy to an insulin concentration that was subsequently normalized by the flow rate through the islet chamber. LOD was taken as the concentration of Ins required to decrease anisotropy to a value lower than 3 times the SD of the blank. For single and grouped islet experiments, LOD was 10 and 20 nM, respectively, due to the different immunoassay reagent concentrations used for the experiments. All error bars are ±1 SD unless indicated otherwise.

### Additional Details for Example 5

Additional details illustrating the optical setup and pressure-driven perfusion system. Mathematical derivations for immunoassay equilibrium equations are provided, as well as a table containing the properties of SeTau-647 and Cy5. Supplementary glucose-stimulated insulin secretion profiles for single and grouped murine in addition to grouped human islets are also shown. Control experiments were conducted to demonstrate insulin secretion was unsynchronized without CCh.

### Derivation for Calculated B/F Vs. [Ins] Calibration Curve

In an insulin competitive immunoassay, the reaction took place as follows:

Ins*+Ins+Ab [symbol] Ab-Ins*+Ab-Ins+Ins*+Ins

The dissociation constant of antibody-insulin complex was defined as:

$${K}_{d} = \frac{[ A b ] [ Ins ]}{[ Ab - Ins ]} \qquad ( 1 )$$

Where, K_{d} is the dissociation constant of antibody-insulin complex. [Ab], [Ins], and [Ab-Ins] are the equilibrium concentration of antibody, insulin, and antibody-insulin complex, respectively. Similarly, K_{d}* was defined as:

$${K}_{d}^{*} = \frac{[ A b ] [ {Ins}^{*} ]}{[ Ab - {Ins}^{*} ]} \qquad ( 1 )$$

Where, K_{d}* is the dissociation constant of antibody-labeled-insulin complex. [Ab], [Ins*], and [Ab-Ins*] were the equilibrium concentration of antibody, labeled-insulin, and antibody-labeled-insulin complex, respectively.

Based on the mass conservation principle, the initial concentration of the labeled-insulin (Ins₀*) was the sum of concentration of species, including insulin* and antibody-insulin* complex. The initial concentration of insulin and antibody could also be written as follows:

Ins₀*=[Ins*]+[Ab−Ins*] (3)

Ins₀=[Ins]+[Ab−Ins] (4)

Ab₀=[Ab−Ins]+[Ab−Ins*]+[Ab] (5)

Where, Ins₀*, Ins₀, and Ab₀ represents the initial concentration of labeled-insulin, insulin, and antibody, respectively.

The bound-to-free ratio (B/F) measured from LIF detector could be defined as:

$$\frac{B}{F} = \frac{[ Ab - {Ins}^{*} ]}{[ {Ins}^{*} ]} \qquad ( 6 )$$

From equations (1)-(6), the bound-to-free ratio can be calculated for a given initial concentration of insulin with a predetermined dissociation constants. For convenience, K_{d} and K_{d}* were set to 10 nM.

### Example Calculation B and F Theoretical B/F

B and F were calculated using equation (3) above, equation (6), and theoretical B/F values obtained using a MATLAB program based on the above derivations.

Assuming Ins₀*=24 nM, as in experimental conditions,

$$[ Ab - {Ins}^{*} ] + [ {Ins}^{*} ] = 24 nM , \qquad ( 3 )$$

Substitute (6) into (3):

1.13F+F=24 nM

### F=11.26 nM

### B=12.74 nM

TABLE 1
Summary of molecular weights and optical properties
of all fluorescently labeled immunoassay products

| Labeled product | MW (kDa) | θ (ns)^{a} | τ (ns) | r₀ |
|---|---|---|---|---|
| Ins*_{Cy-5} | 6.6 | 1.30 | 1.0 | 0.37^{b} |
| Ins*_{Cy-5}-Ab | 156.6 | 31.45 | 1.0 | 0.37^{b} |
| Ins*_{SeTau-647} | 7.6 | 1.50 | 3.2 | 0.40^{c} |
| Ins*_{SeTau-647}-Ab | 157.6 | 31.65 | 3.2 | 0.40^{c} |

^{a}Values were computed using ref³⁴ and the MW for each respective species.
^{b & c}Values were obtained from refs⁴⁵ and ⁴⁶, respectively.

1. A method for insulin detection or monitoring, the method comprising:
providing a sample comprising an islet;
contacting the sample with (i) glucose or (ii) glucose and a balanced salt solution to produce a first stream comprising a target insulin;
contacting the first stream with an anti-insulin antibody and a labeled insulin to produce a second stream comprising the target insulin, an amount of an antibody-bound (B) labeled insulin, and an amount of free (F) labeled insulin; and
determining a ratio of the amount of the antibody-bound (B) labeled insulin to the amount of the free (F) labeled insulin in the second stream, wherein the ratio of B:F is inversely related to a concentration of the target insulin in the second stream.

2. The method of claim 1, wherein the labeled insulin comprises a squaraine rotaxane fluorophore as a label.

3. The method of claim 1, wherein the islet comprises at least one human islet.

4. The method of claim 1, wherein the ratio of B:F is determined homogenously using fluorescence anisotropy.

5. The method of claim 1, wherein the providing of the sample comprises providing a microfluidic device comprising an islet chamber in which the sample is disposed.

6. The method of claim 5, wherein the microfluidic device comprises—
(i) a first fluidic inlet upstream of the islet chamber,
(ii) a second fluidic inlet upstream of the islet chamber,
(iii) a third fluidic inlet downstream of the islet chamber, and
(iv) a fourth fluidic inlet downstream of the islet chamber; and
wherein—
(a) the first stream is a perfusion flow from the islet chamber,
(b) the contacting of the sample with (1) glucose comprises disposing glucose in at least one of the first fluidic inlet and the second fluidic inlet to deliver glucose to the islet chamber, or (2) glucose and a balanced salt solution comprises disposing glucose and the balance salt solution in the first fluidic inlet and the second fluidic inlet, respectively, to deliver glucose and the balanced salt solution to the islet chamber; and
(c) the contacting of the perfusion flow from the islet chamber with the anti-insulin antibody and the labeled insulin comprises disposing the anti-insulin antibody and the labeled insulin in the third fluidic inlet and the fourth fluidic inlet, respectively.

7. The method of claim 6, further comprising disposing carbachol in at least one of the first fluidic inlet and the second fluidic inlet.

8. The method of claim 6, wherein the perfusion flow from the islet chamber has a flow rate of about 0.2 to about 0.5 microliters/minute.

9. The method of claim 5, wherein the microfluidic device is arranged on a metal plate defining one or more voids configured to permit optical detection within a channel of the microfluidic device, and the method further comprises controlling a temperature of the metal plate with one or more heaters, heat sinks, or a combination thereof.

10. The method of claim 1, further comprising contacting an amount of insulin with an amount of a squaraine rotaxane fluorophore to form the labeled insulin.

11. The method of claim 10, wherein a weight ratio of the amount of insulin to the amount of the squaraine rotaxane fluorophore is about 1:1 to about 1.5:1.

12. A method for insulin detection or monitoring, the method comprising:
providing a sample disposed in an islet chamber of a microfluidic device, wherein the microfluidic device comprises—
(i) a first fluidic inlet upstream of the islet chamber, wherein the first fluidic inlet is in fluid communication with a first channel,
(ii) a second fluidic inlet upstream of the islet chamber, wherein the second fluidic inlet is in fluid communication with a second channel, and the first channel and the second channel connect at one or more locations to a first main channel having a first portion upstream of the islet chamber, and a second portion downstream of the islet chamber, and,
(iii) a third fluidic inlet downstream of the islet chamber, wherein the third fluid inlet is in fluid communication with a third channel, and
(iv) a fourth fluidic inlet downstream of the islet chamber, wherein the fourth fluidic inlet is in fluid communication with a fourth channel, and the third channel and the fourth channel connect at one or more locations to the second portion of the main channel to form a second main channel;
disposing glucose in at least one of the first fluidic inlet and the second fluidic inlet to produce a perfusion flow in the second portion of the main channel, wherein the perfusion flow comprises a target insulin;
contacting the perfusion flow with an anti-insulin antibody and a labeled insulin by disposing the anti-insulin antibody and the labeled insulin in the third fluidic inlet and the fourth fluidic inlet, respectively, to produce a stream in the second main channel, wherein the labeled insulin comprises a squaraine rotaxane fluorophore as a label; and
determining a ratio of antibody-bound (B) labeled-insulin to free (F) labeled insulin in the stream in the second main channel, wherein the ratio of B:F is inversely related to a concentration of the target insulin, wherein the ratio is determined homogeneously using fluorescence anisotropy.

13. A method for detecting or monitoring a target, the method comprising:
performing a fluorescence anisotropy-based homogeneous assay of a liquid comprising a labeled target, wherein the labeled target comprises a squaraine rotaxane fluorophore label.

14. A system for detecting or monitoring a target, the system comprising:
a microfluidic device, wherein the microfluidic device comprises an islet chamber, and (i) a first fluidic inlet upstream of the islet chamber, (ii) a second fluidic inlet upstream of the islet chamber, (iii) a third fluidic inlet downstream of the islet chamber, and (iv) a fourth fluidic inlet downstream of the islet chamber;
a metal plate defining one or more voids, wherein the microfluidic device is arranged on the metal plate, and the one or more voids are configured to permit optical detection of a stream within a channel of the microfluidic device;
a first fluid reservoir pressurized with a first flow controller, wherein the first fluid reservoir is in fluid communication with the fourth fluidic inlet.

15. The system of claim 14, further comprising a labeled target disposed in the first fluid reservoir, wherein the labeled target comprises a squaraine rotaxane fluorophore label.

16. The system of claim 15, further comprising a second fluid reservoir pressurized with a second flow controller, optionally a third fluid reservoir pressurized with a third flow controller, and optionally a fourth fluid reservoir pressurized with a fourth flow controller, wherein the second, third, and fourth fluid reservoirs are in fluid communication with different fluidic inlets selected from the first, second, and third fluidic inlets.

17. The system of claim 16, wherein the first, second, third, and/or fourth flow controller is a piezoelectric flow controller.

18. The system of claim 14, further comprising an optical apparatus comprising a laser, a linear polarizer, a dichroic mirror, an emission filter, a polarizing beam splitter, a photomultiplier tube, or a combination thereof.

19. The system of claim 14, wherein the microfluidic device comprises one or more microfluidic channels having a depth of about 80 μm to about 100 μm, a width of about 180 μm to about 220 μm, or a combination thereof.

20. The system of claim 14, wherein—
(i) the first fluidic inlet is in fluid communication with a first channel, the second fluidic inlet is in communication with a second channel, and the first channel and the second channel connect at one or more locations to a first main channel comprising the islet chamber, and
(ii) the third fluid inlet is in fluid communication with a third channel, the fourth fluidic inlet is in fluid communication with a fourth channel, and the third channel and the fourth channel connect with the first main channel at one or more locations downstream of the islet chamber to form a second main channel.
